(19) United States
(12) Patent Application Publication
(10) Pub. No.: US 2020/0131471 A1
(43) Pub. Date: Apr. 30, 2020

(54) METHOD OF MANUFACTURING CELL SPHEROID USING THREE-DIMENSIONAL PRINTING METHOD

(71) Applicant: POSTECH ACADEMY-INDUSTRY FOUNDATION, Pohang-si (KR)

(72) Inventors: Jinah JANG, Pohang-si (KR); Yejin PARK, Gwangyang-si (KR); UiJung YONG, Pohang-si (KR); Sanskrita DAS, Pohang-si (KR); Dong Gyu HWANG, Pohang-si (KR)

(21) Appl. No.: 16/660,924

(22) Filed: Oct. 23, 2019

(30) Foreign Application Priority Data
Oct. 24, 2018 (KR) 10-2018-0127389
Oct. 21, 2019 (KR) 10-2019-0130991

(51) Int. Cl. C12N 5/00; B33Y 10/00; B33Y 70/00; B29C 64/106; B29C 64/386; B33Y 50/00; B33Y 80/00

(52) U.S. Cl. CPC C12N 5/0062; B33Y 10/00; B33Y 70/00; B29C 64/106; B29L 2031/40; B33Y 50/00; B33Y 80/00; C12N 2513/00; B29K 2005/00; B29C 64/386

(57) ABSTRACT

The present invention relates to a method of manufacturing a cell spheroid using three-dimensional bio-printing technology, and the cell spheroid may be used for preventing or treating vascular and endocrine diseases by including mesenchymal stem cells, induced pluripotent stem cells-derived cells, or the like as an active ingredient, or may be used as an in vitro drug testing model.

### TECHNICAL FIELD

The present invention relates to a method of manufacturing a cell spheroid using three-dimensional bio-printing technology, and the cell spheroid may be used for preventing or treating vascular and endocrine diseases comprising mesenchymal stem cells, induced pluripotent stem cells-derived cells, or the like as an active ingredient, or may be used as an in vitro drug testing platform.

### BACKGROUND ART

In vivo, cells grow, differentiate, and multiply by complex interactions in various combinations including extracellular matrix, growth factors, and the like, in a three-dimension. In order to effectively recapitulate this, the conventional two-dimensional cell culture method has its limitations. There are not only morphological differences by proliferation in a single layer, but also there are differences in all aspects, including proliferation, differentiation, difference of secretions, and drug reactivity, and the like. To overcome this, researches on three-dimensional cell culture methods have been actively conducted, and in particular, among them, the three-dimensional spheroid shape can recapitulate the minimum unit of the cell environment in the body, and thus its utilization is very high.

Ischemic disease has a high prevalence of 40%, but shows limitations in treatment of refractory diseases due to limited regeneration of ischemic tissue. There has been a clinical study on the administration through blood vessels using adult stem cells to the development of a therapeutic agent for an ischemic disease so far, but it has been reported that in the conventional treatment, it is difficult to provide the environment that can improve engraftment and proliferation/differentiation of stem cells, and therefore efficiency is low. To solve this problem, hybrid stem cells and three-dimensional spheroid transplantation methods have been developed rather than single stem cell transplantation methods. Such transplantation methods have proved excellent efficiency by animal models of severe cardiovascular disease.

In addition, for treatment of endocrine diseases such as diabetes, etc., a method for effectively transplanting and functioning pancreatic islet cells into the body has emerged, and also, to mimic the form of pancreatic islets, a three-dimensional spheroid is effective.

In addition to prevention and treatment of vascular and endocrine diseases, as well as an in vitro drug testing model, the three-dimensional spheroid can effectively simulate the body and be used as a more accurate drug testing model. In particular, drug screening using animal models also shows limitations due to genetic differences and ethical issues, and therefore it can be used as an alternative to overcome them.

There are two widely used ways of the conventional three-dimensional spheroid manufacturing methods. The first is to put cells in a small U-bottom plate in which cells do not stick to the bottom, to make cells cluster themselves. When using this method, it takes a long time of 24 hours at minimum to 96 hours at maximum, for cells to form a stable spheroid themselves. In addition, various sizes of spheroids are formed according to the degree of cell gathering during manufacture, which does not meet the standard of cell therapeutic agents that require a certain size and a certain effect.

The second method is using a microfluidic device. It is a method that cell contained alginate is intermittently flowed under continuously flowing calcium chloride solution, in which alginate could be crosslinked and formed spheroid shape instantaneously when they meet calcium ion. This method is suitable for producing uniform spheroids, but is likely to fuse with adjacent spheroids before spheroid shape is fully formed, and because the gelation process is dependent on the diffusion time, initially manufactured spheroid shape do not have a constant size and thus it takes time to produce uniform spheroid shape.

It is impossible to control the position of 3 or more axes in both methods, so it is difficult to arrange a 3D structure or the desired location at the same time as the spheroid manufacture. In addition, it is difficult to implement a tissue-specific three-dimensional environment that will provide the microenvironment of cells.

### DISCLOSURE

### Technical Problem

The present invention relates to a method of manufacturing a cell spheroid based on three-dimensional cell printing, which is expected to be used for preventing or treating vascular and endocrine diseases comprising mesenchymal stem cells, induced pluripotent stem cells-derived cells, or the like as an active ingredient, or may be used as an in vitro drug testing platform.

### Technical Solution

Accordingly, the present inventors have developed a three-dimensional cell printing-based technology for manufacturing a three-dimensional spheroid shape of structure, for effective delivery of stem cells to a local site and biological niche microenvironment simulation.

The present invention relates to a method for manufacturing a spheroid based on a three-dimensional cell printing, comprising mesenchymal stem cells, induced pluripotent stem cells-derived cells, or the like as an active ingredient, which is expected to be used for prevention or treatment of vascular and endocrine diseases.

One embodiment of the present invention relates to a method of manufacturing a cell spheroid using a three-dimensional printing method, comprising preparing a bioink composition comprising cells, decellularized extracellular matrix and alginate; and manufacturing a cell spheroid by printing the bioink using the a three-dimensional bio-printing in a micro-extrusion manner The alginate may be used as a gelling polymer.

The manufacturing a spheroid may comprise inserting a nozzle to a mixed solution comprising a gelling agent of the alginate and hydrogel in the chamber; extruding the bioink by applying a pneumatic pressure; and pulling out the injection muzzle from the mixed solution.

The manufacturing a spheroid may comprise applying viscosity to the hydrogel in the chamber by heating a mixed solution comprising a gelling agent of the alginate and hydrogel; inserting a nozzle to the mixed solution comprising a gelling agent of the alginate and hydrogel in the chamber; extruding the bioink by applying a pneumatic pressure; and pulling out the nozzle from the mixed solution. The mixed solution may have the viscosity so that the extruded bioink are detached by pulling out the nozzle to form a cell spheroid. Accordingly, the mixed solution may be heated so that the hydrogel has an appropriate viscosity.

Herein, the ‘bioink’ is a generic term for materials which comprise living cells or bio-molecules, and can manufacture a required structure by applying it to a bioprinting technology. The bioink of the present invention includes a liquid, semi-solid or solid composition comprising a plurality of cells. Accordingly, the bioink should provide physical properties for three-dimensional processing and a biological environment for cells to perform targeted a function. It is preferable that supply of nutrients and oxygen required for survival of cells in the ink extruding member is conducted properly when the printing process is long. In addition, cells should be protected from physical stresses occurring in the printing process. Besides, the bioink should have physical properties required in the printing process such as appropriates viscosity without blockage of the nozzle, for enhancing repeatability and productivity of three-dimensional patterning. The ink according to the present invention is preferably a hydrogel, and accordingly, the ink may comprise a gelling polymer, and for example, it may comprise one or more kinds selected from the group consisting of a gelling polymer, a cell, a growth factor and extracellular matrix.

The present invention also provides a bioink composition, wherein the bioink composition further comprises a tissue-derived ingredient. The tissue-derived ingredient means the gelated materials that contain the decellularized extracellular matrix derived from the certain tissues of animals such as cartilage, kidney, heart, liver, muscle or the like, as a main ingredient, and this may be contained to intensify tissue specificity of the bioink composition.

The bioink composition according to one embodiment of the present invention may further comprise a cell culture medium. The cell culture medium is a concept of comprising any medium suitable for targeted cells.

According to one embodiment of the present invention, the mixing ratio of the solution comprising cells and decellularized extracellular matrix, and the solution of alginate may be 1:0.33 to 1:3 in a volume ratio, and then, the decellularized extracellular matrix may be comprised as a decellularized extracellular matrix solution at a concentration of 2.6% by weight, and the alginate may be comprised as an alginate solution at a concentration of 2% by weight.

The decellularized extracellular matrix used in the bioink composition of the present invention may be a solution having a concentration of decellularized extracellular matrix of 0.5 to 5% by weight, and the alginate solution may be a solution having a concentration of alginate of 0.5 to 5% by weight. The bioink composition comprises cells, decellularized extracellular matrix and alginate. The alginate may be used as a gelling polymer.

The decellularized extracellular matrix solution and the alginate solution may comprise the decellularized extracellular matrix and alginate in a specific concentration, respectively, so that the decellularized extracellular matrix and alginate have an appropriate concentration ratio when mixed and comprised in the bioink composition.

The concentration ratio of the decellularized extracellular matrix and alginate comprised in the bioink composition may be more than 1:0.5 to 1:5, more than 1:0.5 to 1:4, more than 1:0.5 to 1:3.5, more than 1:0.5 to 1:3, more than 1:0.5 to 1:2.5, more than 1:0.5 to 1:2, more than 1:0.5 to 1:1.5, more than 1:0.5 to 1:1.4, more than 1:0.5 to 1:1.3, more than 1:0.5 to 1:1.2, more than 1:0.5 to 1:1.1, more than 1:0.5 to 1:1, 1:0.6 to 1:5, 1:0.6 to 1:4, 1:0.6 to 1:3.5, 1:0.6 to 1:3, 1:0.6 to 1:2.5, 1:0.6 to 1:2, 1:0.6 to 1:1.5, 1:0.6 to 1:1.4, 1:0.6 to 1:1.3, 1:0.6 to 1:1.2, 1:0.6 to 1:1.1, 1:0.6 to 1:1, 1:0.6 to 1:5, 1:0.6 to 1:4, 1:0.6 to 1:3.5, 1:0.6 to 1:3, 1:0.6 to 1:2.5, 1:0.6 to 1:2, 1:0.6 to 1:1.5, 1:0.6 to 1:1.4, 1:0.6 to 1:1.3, 1:0.6 to 1:1.2, 1:0.6 to 1:1.1, 1:0.6 to 1:1, 1:0.7 to 1:5, 1:0.7 to 1:4, 1:0.7 to 1:3.5, 1:0.7 to 1:3, 1:0.7 to 1:2.5, 1:0.7 to 1:2, 1:0.7 to 1:1.5, 1:0.7 to 1:1.4, 1:0.7 to 1:1.3, 1:0.7 to 1:1.2, 1:0.7 to 1:1.1, 1:0.7 to 1:1, 1:0.8 to 1:5, 1:0.8 to 1:4, 1:0.8 to 1:3.5, 1:0.8 to 1:3, 1:0.8 to 1:2.5, 1:0.8 to 1:2, 1:0.8 to 1:1.5, 1:0.8 to 1:1.4, 1:0.8 to 1:1.3, 1:0.8 to 1:1.2, 1:0.8 to 1:1.1, 1:0.8 to 1:1, 1:0.9 to 1:5, 1:0.9 to 1:4, 1:0.9 to 1:3.5, 1:0.9 to 1:3, 1:0.9 to 1:2.5, 1:0.9 to 1:2, 1:0.9 to 1:1.5, 1:0.9 to 1:1.4, 1:0.9 to 1:1.3, 1:0.9 to 1:1.2, 1:0.9 to 1:1.1, 1:0.9 to 1:1, 1:1 to 1:5, 1:1 to 1:4, 1:1 to 1:3.5, 1:1 to 1:3, 1:1 to 1:2.5, 1:1 to 1:2, 1:1 to 1:1.5, 1:1 to 1:1.4, 1:1 to 1:1.3, 1:1 to 1:1.2, or 1:1 to 1:1.1.

For example, the decellularized extracellular matrix solution may comprise the decellularized extracellular matrix in a concentration of 0.1 to 5% by weight, 0.1 to 4% by weight, 0.1 to 3% by weight, 0.1 to 2.5% by weight, 0.1 to 2% by weight, 0.5 to 5% by weight, 0.5 to 4% by weight, 0.5 to 3% by weight, 0.5 to 2.5% by weight, 0.5 to 2% by weight, 1 to 5% by weight, 1 to 4% by weight, 1 to 3% by weight, 1 to 2.5% by weight, 1 to 2% by weight, 1.5 to 5% by weight, 1.5 to 4% by weight, 1.5 to 3% by weight, 1.5 to 2.5% by weight, 1.5 to 2% by weight, 2 to 5% by weight, 2 to 4% by weight, 2 to 3% by weight, or 2 to 2.5% by weight.

For example, the alginate solution may comprise alginate in a concentration of 0.1 to 5% by weight, 0.1 to 4% by weight, 0.1 to 3% by weight, 0.1 to 2.5% by weight, 0.1 to 2% by weight, 0.5 to 5% by weight, 0.5 to 4% by weight, 0.5 to 3% by weight, 0.5 to 2.5% by weight, 0.5 to 2% by weight, 1 to 5% by weight, 1 to 4% by weight, 1 to 3% by weight, 1 to 2.5% by weight, 1 to 2% by weight, 1.5 to 5% by weight, 1.5 to 4% by weight, 1.5 to 3% by weight, 1.5 to 2.5% by weight, 1.5 to 2% by weight, 2 to 5% by weight, 2 to 4% by weight, 2 to 3% by weight, or 2 to 2.5% by weight.

For example, the concentration of the decellularized extracellular matrix comprised in the bioink composition, or the concentration of the alginate comprised in the bioink composition may have a lower limit value of 0.01% by weight, 0.05% by weight, 0.1% by weight, 0.2% by weight, 0.3% by weight, 0.4% by weight, 0.5% by weight, 0.6% by weight, 0.7% by weight, 0.8% by weight, 0.9% by weight, or 1% by weight, and a upper limit value of 10% by weight, 9% by weight, 8% by weight, 7% by weight, 6% by weight, 5% by weight, 4% by weight, 3% by weight, 2% by weight, 1.5% by weight, 1.4% by weight, 1.3% by weight, 1.2% by weight, 1.1% by weight, or 1% by weight, and the concentration of the decellularized extracellular matrix comprised in the bioink composition, or the concentration of the alginate comprised in the bioink composition may be determined by the combination of the lower limit value and the upper limit value. For example, the concentration of the decellularized extracellular matrix comprised in the bioink composition, or the concentration of the alginate comprised in the bioink composition may be 0.1 to 5% by weight, 0.1 to 3% by weight, 0.1 to 2% by weight, 0.1 to 1.5% by weight, 0.5 to 5% by weight, 0.5 to 3% by weight, 0.5 to 2% by weight, or 0.5 to 1.5% by weight, and as an example, it may be 1% by weight.

The cell of the present invention may be a cell available for manufacturing the cell spheroid, and may include mesenchymal stem cells, induced pluripotent stem cell-derived cells, progenitor cells, and the like, and for example, may be heart cells, cells capable of being differentiated into vascular cells, islet cells and induced pluripotent stem cell-based insulin producing cells.

The bioink according to the present invention may comprise cells, and applicable cells or tissue is not particularly limited, and it may be one or more selected from the group consisting of stem cell, osteoblast, myoblast, tenocyte, neuroblast, fibroblast, glioblast, germ cell, hepatocyte, renal cell, Sertoli cell, chondrocyte, epithelial cell, cardiovascular cell, keratinocyte, smooth muscle cell, cardiomyocyte, glial cell, endothelial cell, hormone secreting cell, immunocyte, pancreatic islet cell and neuron. Specifically, CPC (cardiac progenitor cell), EPC (endothelial progenitor cell), human islet cell, vascular cell, neuron, immunocyte, or the like may be used.

Herein, the “spheroid” is a small ‘sphere’ structure produced through tertiary tissue culture, and the spheroid is a kind of small ‘tissue’ and may divide its functions by separating the exterior and the interior, and has a form of replicating a functional unit of tissue. The spheroid culture has been developed as a platform which has a similar intrinsic form or property of an animal or human tissue, and also can be utilized for research by applying it.

In the three-dimensional printing method for cell spheroid manufacture according to the present invention, spheroids in a size of 100 to 500 μm may be manufactured by performing under the condition of the time of applying a pneumatic pressure of 0.01 to 0.1 second. In addition, a nozzle size of a printer used for the three-dimensional printing method may be 25 to 30 Gauge, for example 27 Gauge. For example, the nozzle size may be 27 Gauge, and printing may be performed under the pneumatic pressure condition of 30 to 40 kPa. Furthermore, as a gelling agent for instant cross-linking, a calcium chloride solution may be used as mixed with Pluronic F-127 hydrogel (20% by weight) having high viscosity, and after producing spheroids, various buffers for washing the gelling agent, for example, a mixed solution in which HBSS buffer and calcium chloride solution are mixed, may be used.

The cell spheroid manufactured according to the method of the present invention has characteristics of being mixed with one kind or various kinds of cells and capable of implementing a niche environment of tissue to be regenerated. In addition, according to the present invention, a plurality of spheroids may be formed by one process, specifically, in which makes a program code including a trigonometrical function to form a plurality of spheroids in an equal interval, and locates a nozzle at a point where spheroids are prepared according to the determined radius of the circle and the number of spheroids. In addition, in order to prepare the spheroid continuously, it is a method of printing 3,000 or more spheroids at maximum in one batch along the spiral track formed by decreasing the radius after passing through the half-track of circle.

The method of manufacturing a cell spheroid according to one embodiment of the present invention may make a plurality of cell spheroids, for example, 2 or more, 10 or more, 50 or more, 100 or more, 300 or more, 500 or more, 800 or more, 1000 or more, 1500 or more, 2000 or more, 2500 or more, or 3000 or more of cell spheroids at one time by extruding and printing the bioink continuously.

The injection nozzle may move along the spiral track, and the cell spheroid may be located along the spiral track. In addition, the cell spheroid may be located on circumferences of at least two concentric circles

The present invention raises a need of research on various manufacture technologies to maximize the efficacy of stem cells, to solve the problem of the aforementioned methods using a U-shaped plate or a microfluid device as the conventional three-dimensional spheroid manufacture methods. In particular, the 3D bio-printing technology, which can locate optimal cell conditions, biomaterials and extracellular matrix and the like separately, depending on the tissue to be regenerated, is determined as an optimal technology which can overcome these limitations. The spheroid manufactured by using bio-printing forms three-dimensional spheroid immediately after manufacturing process, and have advantages that spheroids can be manufactured with the same size under the same condition consecutively, since it is a method that forms a three-dimensional spheroid immediately after passing through a manufacture process and forms it by distributing bioink in the same volume, not by self-assembly of cells. In addition, the spheroid has a possibility to manufacture a three-dimensional structure consisting of spheroids by driving heads of x, y and z axes while distributing.

Specifically, in one embodiment of the present invention, manufacturing a cell spheroid by printing the bioink may comprise determining information of a radius of a semicircle having the longest radius among the spiral tracks of the injection nozzle, the number of spheroids located in the circumference of the semicircle having the longest radius among the spiral tracks, a radius of a semicircle having the shortest radius among the spiral tracks, and an interval between circumferences of the spiral tracks; determining the location at which the bioink is sprayed by the injection nozzle using the determined information; and preparing continuously a plurality of cell spheroids by printing the bioink on the determined locations. The plurality of cell spheroids may be manufactured apart at a certain interval, and the certain interval may be determined by the radius of the semicircle having the longest radius among the spiral tracks and the number of spheroids located on the circumference of the semicircle having the longest radius of the spiral tracks.

More specifically, the method of manufacturing a cell spheroid according to one embodiment of the present invention will be described with FIG. 1. A mixed solution was obtained by mixing the calcium chloride solution (0.1 M) as a gelling agent of alginate, and Pluronic F-127 solution (20% by weight) that is a hydrogel having temperature sensitive viscosity property and then it was put on a petri dish. The petri dish was located on the bottom stage of printer head at a temperature of 25° C. Next, the nozzle was inserted into the inside of the mixed solution on the position to prepare spheroids and extruded the bioink by applying a pneumatic pressure to produce spheroids. Then, when the nozzle is raised up (pulled up), and the spheroids are left only (separated) in the mixed solution having viscosity, and the nozzle is pulled out. Then, in order to prevent drops from tangling with each other when drops are formed on the same position, the printer head is moved to the next position and can prepare spheroids by the same way.

The mixed solution may have appropriate viscosity so that the bioink extruded from the injection nozzle is separated from the injection nozzle, when the injection nozzle is pulled out. Accordingly, the mixed solution may be heated have appropriate viscosity, for example, at 10 to 50° C., 10 to 40° C., 10 to 30° C., 20 to 50° C., 20 to 40° C., or 20 to 30° C. As the mixed solution is heated, the viscosity of hydrogels in the mixed solution is increased so as to increase the viscosity of the mixed solution.

The movement of the dispenser may be controlled to manufacture several spheroids. The number of spheroids to be manufactured in one batch is determined, and G-codes designating each position value are made. To make G-codes automatically, a code generator was made using python. If the initial radius to start, the last radius and the number of spheroids to be manufactured are inserted to the code generator, G-codes are automatically produced. The movement distance of the dispenser is obtained by using a trigonometrical function depending on the number of spheroids, and codes are generated to prepare spheroids on a specific position in a chamber. In addition, to print spheroids effectively, after passing through the half-track of circle, it can produce spheroids while rotating in a spiral track, by decreasing the radius to a certain length and the number of spheroids.

After verifying an accuracy of the code by simulation of whether the printing process may run well, spheroids are manufactured. The simulation uses a program called CAMotics confirming G-codes virtually, and it represents the moved track in a green solid line (FIG. 3a). The printing code starts from the starting point of coordinate system and goes to the chamber. Then, the code performs according to the specific radius and the number of spheroids in the chamber. After calculating a point at a certain interval so as to produce spheroids uniformly in the circle, when it arrives at the point, it goes down in a Z-axis direction to produce spheroids with pneumatic pressure ON/OFF modes and then comes up again and heads to the next point.

FIG. 2 describes the method of calculating the position to manufacture spheroids. When the initial radius is set to 23 mm and the number of spheroids is set to 90, the spheroids are produced by dividing the semicircle with the radius of 23mm into 90 parts in a code and moving at each point in a z-axis direction. After completing the track of semicircle, the spheroids are produced at each point by decreasing the radius by 0.3 mm and minus 1 from the number of spheroids, and dividing the semicircle with the radius of 22.7 mm into 89 parts. At each time when the track of semicircle passes, the radius and the number of spheroids is decreased so as to produce spheroids, until reaching the final radius.

The present invention established the basic production mechanism of manufacturing a spheroid, the mixing requirement of the bioink composition comprised in a spheroid, and the three-dimensional bio-printing process condition for manufacturing a spheroid in a size being proper for using purpose.

At first, z-direction movement can be controlled to locate the nozzle in the chamber containing the calcium chloride solution (0.1 M) and Pluronic F-127 solution (20% by weight) placed on the bottom (x-y plane). Then, the droplets in a spherical shape are produced by on/off control of the pneumatic pressure of the dispenser in a short time. The time of applying the pneumatic pressure can control the size of spheroids. Then, the nozzle is pulled out of the solution, so that spheroids are separated from the nozzle and submerged in the chamber.

The alginate aqueous solution that is one material comprised in the spheroids may maintain the shape of the spheroid, because it has a gelling property at contacting with a calcium ion of the calcium chloride solution.

The parameters may be determined depending on the properties of materials contained in the spheroids. Since the method of manufacturing a spheroid in the present invention is to implement an in vivo microenvironment, the mixture of decellularized extracellular matrix and cells may be used as materials to prepare the spheroid. Accordingly, the bioink of the present invention comprises cells, decellularized extracellular matrix and alginate. The alginate may be used as a gelling polymer.

Because it is difficult for only decellularized extracellular matrix to induce an instant cross-linking only decellularized extracellular matrix, the alginate hydrogel is mixed and used. In as aspect of the shape of spheroid, when the ratio of the alginate hydrogel is higher than the decellularized extracellular matrix, it is easy to maintain the sphere shape of spheroid, but the cell affinity and cell adhesiveness are relatively lowered. On the contrary to this, when the alginate ratio is low, the instant cross-linking of the final mixed bioink is not conducted, and there is a problem that it is difficult to print the spheroid in a sphere shape due to the higher viscosity of the decellularized extracellular matrix than the alginate. As a result of verifying various conditions, it has been confirmed that it is the most appropriate condition when the concentration ratio of the decellularized extracellular matrix (mixed with cells) and the alginate is more than 1:0.5 to 1:5, for example, about inside and outside 1:1.

In the method of manufacturing a cell spheroid according to the present invention, the time of applying a pneumatic pressure to a three-dimensional printer is about between 0.01-0.1 second, and the size of the spheroid is manufactured largely in proportion to the time of applying the pneumatic pressure. The manufactured spheroid size has a diameter of about 300-500 μm, to be properly delivered by syringe. The spheroid has no limitation on the size as a building block forming a three-dimensional shape. However, the printing is easy, and the viability of cells in the cell spheroid is high after culturing for a while, when the size based on the diameter is 300 to 500 μm, but there is no limitation on the size.

Since spheroids are manufactured continuously on only one position in case of the conventional method of manufacturing a spheroid using a microfluid, the spheroids cannot be manufactured in the same size, because the spheroids adhere together in the cross-linking process. However, the cell spheroid manufactured by the three-dimensional printing method of the present invention is produced to distribute the spheroids in one dish evenly, as the printer head moves continuously. The method of present invention has an advantage of conducting the cross-linking process of many spheroids in the same size.

In the three-dimensional printing method for manufacturing a cell spheroid according to the present invention, the spheroids in a size of 300 to 500 μm may be manufactured by performing under the condition that the time of applying a pneumatic pressure is 0.01 to 0.1 second. In addition, the nozzle size of the printer used in the three-dimensional printing method may be 25 to 28 Gauge, for example, 27 Gauge. For example, it may perform printing under the condition that the nozzle size is 27 Gauge and the pneumatic pressure is 30 to 40 kPa. When the nozzle size becomes bigger (the Gauge becomes smaller) or the pneumatic pressure is increased, the spheroid size becomes bigger gradually or becomes smaller under the opposite condition.

When the pressure (pneumatic pressure) applied to the printing head (dispenser) is too low, it is difficult to take the standardized process. When the pressure is high, the size of the capsule for transplantation becomes too large. The smaller nozzles also make it difficult to take the standardized process, and the larger size makes the capsule for transplantation larger, thereby being inadequate for manufacturing injectable spheroids such as a catheter in the future. When the pneumatic pressure of about 5-100 kPa was tested under the condition of the nozzle of 27 Gauge, to confirm the possibility of manufacturing a capsule stably, the production efficiency was the best under the condition of about 30-40 kPa.

The cell in the spheroid form according to the present invention may be used for treatment of ischemic vascular diseases or as a structure of delivering islet cells, and various delivery methods such as an injection form or a three-dimensional structure form, or the like may be applied, and it may be used as an in vitro drug testing platform.

Another embodiment of the present invention relates to a composition for manufacturing a cell spheroid using a three-dimensional printing method, comprising cells, decellularized matrix and alginate. The alginate may be used as a gelling polymer.

The concentration ratio of the decellularized extracellular matrix and the alginate in the composition may be more than 1:0.5 to 1:5, more than 1:0.5 to 1:4, more than 1:0.5 to 1:3.5, more than 1:0.5 to 1:3, more than 1:0.5 to 1:2.5, more than 1:0.5 to 1:2, more than 1:0.5 to 1:1.5, more than 1:0.5 to 1:1.4, more than 1:0.5 to 1:1.3, more than 1:0.5 to 1:1.2, more than 1:0.5 to 1:1.1, more than 1:0.5 to 1:1, 1:0.6 to 1:5, 1:0.6 to 1:4, 1:0.6 to 1:3.5, 1:0.6 to 1:3, 1:0.6 to 1:2.5, 1:0.6 to 1:2, 1:0.6 to 1:1.5, 1:0.6 to 1:1.4, 1:0.6 to 1:1.3, 1:0.6 to 1:1.2, 1:0.6 to 1:1.1, 1:0.6 to 1:1, 1:0.6 to 1:5, 1:0.6 to 1:4, 1:0.6 to 1:3.5, 1:0.6 to 1:3, 1:0.6 to 1:2.5, 1:0.6 to 1:2, 1:0.6 to 1:1.5, 1:0.6 to 1:1.4, 1:0.6 to 1:1.3, 1:0.6 to 1:1.2, 1:0.6 to 1:1.1, 1:0.6 to 1:1, 1:0.7 to 1:5, 1:0.7 to 1:4, 1:0.7 to 1:3.5, 1:0.7 to 1:3, 1:0.7 to 1:2.5, 1:0.7 to 1:2, 1:0.7 to 1:1.5, 1:0.7 to 1:1.4, 1:0.7 to 1:1.3, 1:0.7 to 1:1.2, 1:0.7 to 1:1.1, 1:0.7 to 1:1, 1:0.8 to 1:5, 1:0.8 to 1:4, 1:0.8 to 1:3.5, 1:0.8 to 1:3, 1:0.8 to 1:2.5, 1:0.8 to 1:2, 1:0.8 to 1:1.5, 1:0.8 to 1:1.4, 1:0.8 to 1:1.3, 1:0.8 to 1:1.2, 1:0.8 to 1:1.1, 1:0.8 to 1:1, 1:0.9 to 1:5, 1:0.9 to 1:4, 1:0.9 to 1:3.5, 1:0.9 to 1:3, 1:0.9 to 1:2.5, 1:0.9 to 1:2, 1:0.9 to 1:1.5, 1:0.9 to 1:1.4, 1:0.9 to 1:1.3, 1:0.9 to 1:1.2, 1:0.9 to 1:1.1, 1:0.9 to 1:1, 1:1 to 1:5, 1:1 to 1:4, 1:1 to 1:3.5, 1:1 to 1:3, 1:1 to 1:2.5, 1:1 to 1:2, 1:1 to 1:1.5, 1:1 to 1:1.4, 1:1 to 1:1.3, 1:1 to 1:1.2, or 1:1 to 1:1.1.

### ADVANTAGEOUS EFFECTS

The present invention relates to a method of manufacturing a cell spheroid using three-dimensional bio-printing technology, and the cell spheroid may be used for preventing or treating vascular and endocrine diseases comprising mesenchymal stem cells, induced pluripotent stem cells-derived cells, or the like as an active ingredient, or may be used as an in vitro drug testing platform.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a schematic diagram of the process of manufacturing spheroids according to one embodiment of the present invention.

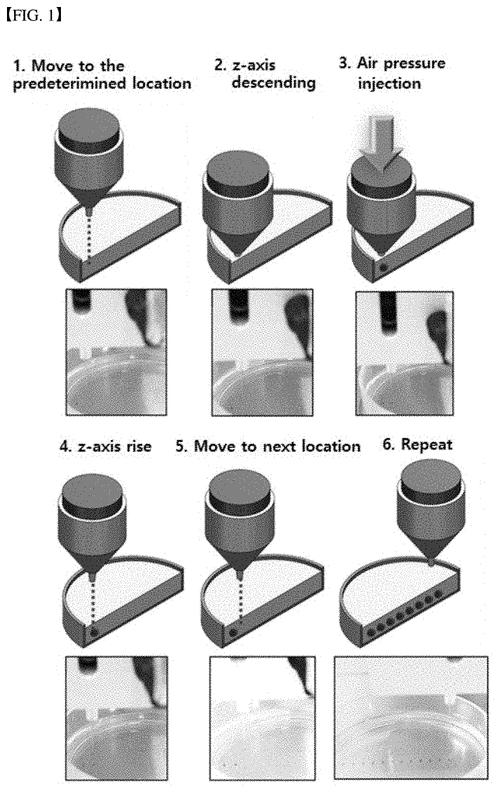

FIG. 2 is a schematic diagram showing the method of calculating the location where to produce spheroids according to one embodiment of the present invention.

FIG. 3a is schematic diagram showing the result of printing simulation using the CAMotics program which virtually confirms a G-code according to one embodiment of the present invention.

FIG. 3b is a drawing which shows the track of the spheroid manufactured by printing the bioink composition according to one embodiment of the present invention.

FIG. 4 is the experimental result showing the result of manufacturing a cell spheroid according to the mixing ratio of extracellular matrix and alginate which constitutes the bioink composition according to one embodiment of the present invention.

FIG. 5 is capsule photographs showing the result of printing under various nozzle and pneumatic pressure conditions according to one embodiment of the present invention.

FIG. 6a is a photograph showing the viability of cells in the cell spheroid manufactured according to one embodiment of the present invention over time, and FIG. 6b shows it divided by cell.

### DETAILED DESCRIPTION OF THE EMBODIMENTS

The present invention will be described in more detail by the following examples, but the present invention is not intended to be limited by the following exemplary examples.

### Example 1

### Cell Spheroid Manufacture

### (1) Bioink Manufacture

Bioink for manufacturing spheroids comprised cells, decellularized extracellular matrix and alginate. A sufficient number of cells should be secured to manufacture cell spheroids. The volume ratio of the cell suspension in which the decellularized extracellular matrix and cells comprised in bioink were mixed was 1:1. The decellularized extracellular matrix was a solution at a concentration of 2.6% by weight, and as the alginate solution, a solution at a concentration of 2% by weight was used.

As cells comprised in bioink, cardiac progenitor cells (CPC), endothelial progenitor cells (EPC), or cells mixing cardiac progenitor cells and endothelial progenitor cells at the same number ratio were used. The decellularized extracellular matrix obtained from swine heart tissue was used as the decellularized extracellular matrix of the bioink. The cell concentration used was all 10⁷ cells/mL, and in case of the mixed cells, cardiac progenitor cells (CPC) and endothelial progenitor cells (EPC) were mixed in equal numbers so that the total number of cells was 10⁷ cells/mL.

When printing once, cells were used in an amount of 10⁷ cells/mL, and decellularized extracellular matrix solution 200 ul, cell culture medium 60 ul, and alginate solution 260 ul were used, and thereby bioink in a volume of 520 ul in total was prepared. Then, the concentration of the decellularized extracellular matrix in the decellularized extracellular matrix solution and suspension of cell culture medium was 2.0% by weight, and it was same as the alginate use of the alginate solution of 2.0% by weight. Accordingly, the concentration of the decellularized extracellular matrix and the concentration of the alginate in the mixed solution in which the decellularized extracellular solution, cell culture medium and alginate solution were mixed were same as 1.0% by weight. After detaching cells from the dish using trypsin, they were mixed to the bioink in which alginate and decellularized extracellular matrix were mixed evenly. Then, care was taken to avoid bubbles, and since the decellularized extracellular matrix is not gelated during the experiment when being used under 4° C., ink was mixed in an ice-box.

### (2) Bioink Printing

Bioink in which all substances were evenly mixed was put in a 3 ml syringe with a 27 G nozzle, and this was connected to the printer nozzle area. As a chamber, a petri dish was used, and the mixed solution of calcium chloride solution (0.1 M) and Pluronic F-127 solution (20% by weight) was added by 5 ml, and then to increase the viscosity of PF-127 solution, the temperature was set to 25° C.

When inputting the center of the water tank to the first part of the code, a cell spheroid is manufactured by drawing a circle around the position. The nozzle moved to the center of the bath, and when it reached the position corresponding to the same distance as the length of the radius from the center, the nozzle was lowered down so that the tip of the nozzle was submerged in the solution in the tank, and the pneumatic pressure was applied to form a spheroid at the tip of the nozzle. Then, when the nozzle was raised again, due to the viscosity of PF-127, the spheroid was not raised and was separated, being immersed in the chamber. The pneumatic pressure time was 0.032 seconds, and the pneumatic pressure was 30 kPa.

### Example 2

### Spheroid Manufacture According to Pneumatic Pressure Time Change

In order to investigate the effect according to the pneumatic pressure time, spheroids were manufactured under various conditions, and spheroids having various sizes according to the change of the pneumatic pressure time could be manufactured.

Specifically, bioink was manufactured by the same method as Example 1 to connect it to the printer nozzle. By increasing the time of applying the pneumatic pressure from the initial pneumatic pressure time 0.032 seconds to manufacture spheroids. To form spheroid at the end of the nozzle on the code, when applying the pneumatic pressure, the delay time was set to increase the pneumatic pressure time. By setting the pneumatic pressure time to 0.1 second, 0.3 seconds, 0.5 seconds and 0.7 seconds, spheroids were manufactured. The result was shown in Table 1.

TABLE 1

| | Pneumatic pressure time (sec) | | | | |
|---|---|---|---|---|---|
| | 0.032 | 0.1 | 0.3 | 0.5 | 0.7 |
| Diameter of | 372.24 | 476.67 | 530.34 | 734.83 | 1047.06 |
| spheroid (um) | | | | | |

As a result, in the range of the pneumatic pressure time of 0.01 to 0.2 seconds (for example, 0.032 seconds), spheroid having a diameter of 300 to 500 μm were manufactured, and when increasing the time of applying the pneumatic pressure to 0.7 seconds or more, spheroid having a size of 1000 μm or more were manufactured.

### Example 3

### Consecutive Manufacture of Spheroids

To manufacture a large number of spheroids consecutively, bioink was distributed while the injection nozzle moved along the circumferences of concentric circles with a certain interval, and spheroids were consecutively manufactured in quantity.

Specifically, to manufacture a large number of spheroids at a certain interval, it was considered that it was the most effective that the injection nozzle moved along the track of circles, and accordingly, codes were produced so that the injection nozzle could move the circle track. In addition, it was not that the injection nozzle moved to one circle track and the spheroid manufacture was completed, but bioink was distributed along the concentric circles following the circle track with gradually decreasing radius, thereby allowing massive production of spheroids.

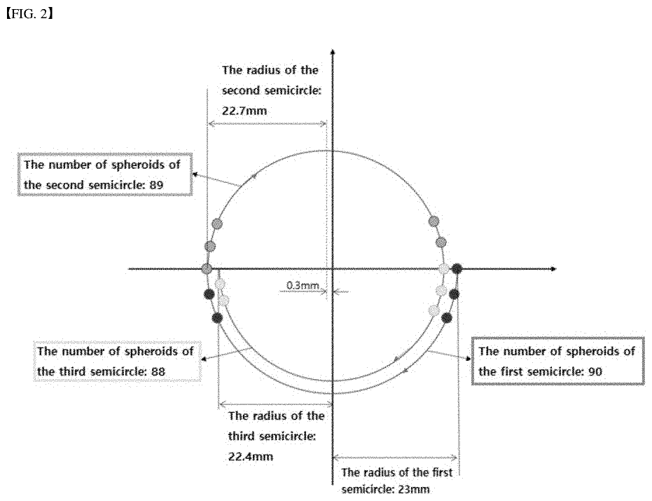

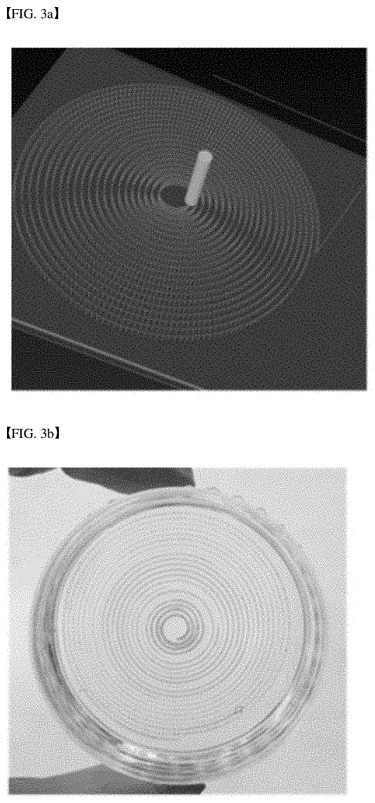

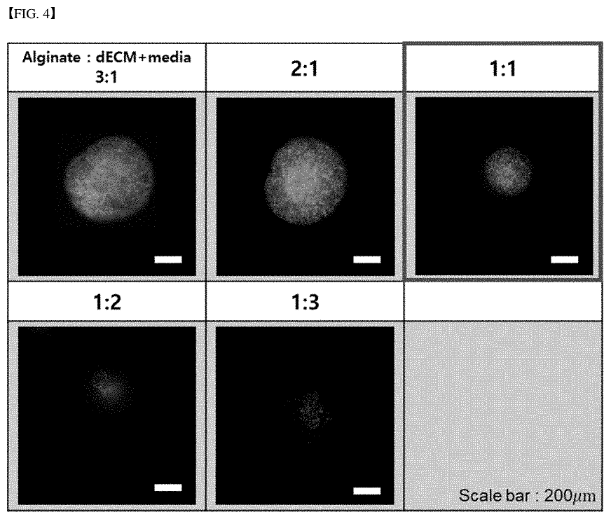

For example, after locating the nozzle on the position of the center of circles at first, the initial radius (23 mm) and the number of spheroids to be manufactured (90), the final radius (5.6 mm), and the interval between circumferences (0.3 mm) were substituted on codes, and then each point where spheroids were located was drawn using a trigonometric function. When the injection nozzle reached to each distributing point, after lowering the nozzle down as Example 1, the pneumatic pressure was distributed to manufacture spheroids. When distribution was completed, the nozzle rose up again, and immediately moved to the next position using the difference between the next position and the present position, and then bioink was distributed in the same manner to manufacture spheroids. When turning around all the track of the semicircle by repeating this process consecutively, by decreasing one of the number of spheroids on the circumference with the radius of 22.7 mm which was reduced by 0.3 mm from the initial radius, the point where the nozzle should be positioned was drawn using a trigonometrical function so as to manufacture 89 spheroids. When 89 spheroids were manufactured all while turning around the semicircle, the distribution position was drawn to produce 88 spheroids on the 22.4 mm circle with the radius reduced by 0.3 mm again. The above process was repeated until the smallest radius of the concentric circle reached 5.6 mm, and as a result, 3000 or more of spheroids could be automatically manufactured in one batch. FIG. 2 is a drawing which shows a spiral shape of track which can consecutively manufacture spheroids while decreasing the radius of the semicircle and the number of spheroids according to one example of the present invention. FIG. 3a is a schematic diagram showing the result of printing simulation using a program called CAMotics confirming G-code virtually according to one example of the present invention, and FIG. 3b is a photograph of the track of spheroids with actual printing.

### Example 4

Spheroid Manufacture According to the Mixing Ratio of Decellularized Extracellular Matrix (dECM) and Alginate

To control the bioink composition, it was performed by the substantially same method as (1) of Example 1, but bioink was manufactured so as to comprising no cells and comprising decellularized extracellular matrix and alginate at various concentration ratios.

Specifically, cells were not used, and only the suspension comprising decellularized extracellular matrix and the alginate were used. For suspension manufacture, the decellularized extracellular matrix solution (concentration: 2.6%) of 400 ul was added to the cell culture medium of 120 ul, and they were mixed homogeneously to prevent bubbles. By the mixing, the concentration of the decellularized extracellular matrix of the suspension was 2.0% by weight. This was divided to 5 groups and 100 ul was put in an e-tube each.

The alginate solution (concentration: 2%) was mixed evenly in each tube by adding 300 ul, 200ul, 100 ul, 50 ul, or 33.33 ul, respectively, so that the concentration of the decellularized extracellular matrix and the concentration of the alginate were 1:3, 1:2, 1:1, 1:0.5, and 1:0.33, and the mixed volume ratio of the suspension and the alginate solution was 1:3, 1:2, 1:1, 1:0.5, and 1:0.33.

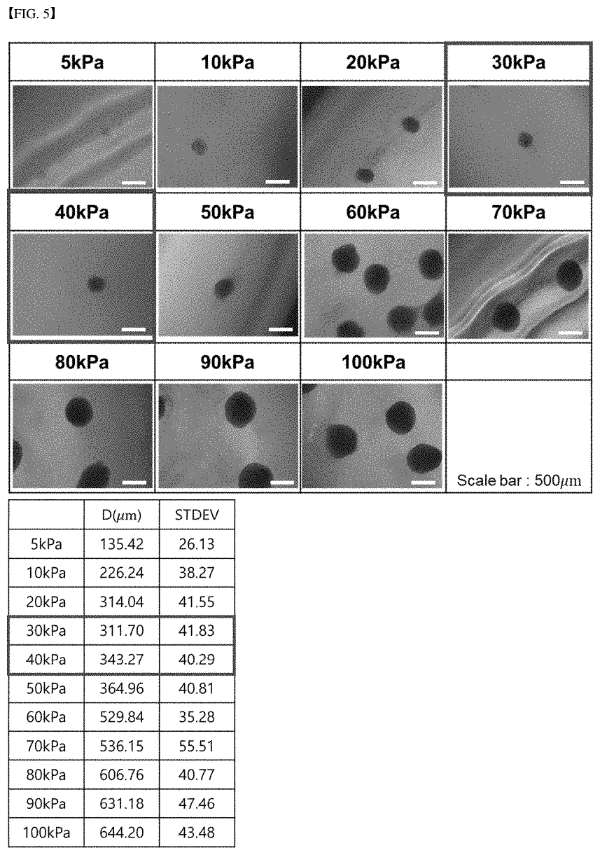

The bioink was moved to a syringe one by one in turn and was connected to the printer nozzle. Since the composition became heterogeneous over time when the bioink was moved to the syringe in advance, it was stored in the e-tube and was moved to a syringe just before printing. All was printed under the same printing condition, and thereby the shape of the spheroid found the homogeneous ratio. Eight spheroids were manufactured along the straight track by each experiment, and the average shape was examined The result was shown in FIG. 4.

FIG. 4 is the experimental result showing the result of cell spheroid manufacture according to the concentration ratio of decellularized extracellular matrix and alginate composing the bioink composition according to one example of the present invention. As shown in FIG. 4, when the concentration ratio of decellularized extracellular matrix and alginate was 1:3, 1:2, and 1:1, that is the concentration of alginate was more than the concentration of decellularized extracellular matrix concentration, the shape of spheroids was homogeneous. At this point, the cell affinity increased as the ratio of the alginate was low, and therefore it could be seen that the concentration ratio of the decellularized extracellular matrix and alginate of 1:1 to 1:2, for example, the concentration ratio of 1:1 was suitable for spheroid manufacture.

### Example 5

### Spheroid Manufacture According to Various Pneumatic Pressure Conditions

To investigate the change according to the pneumatic pressure during spheroid manufacture, it was performed by the substantially same method as Example 1, but bioink was manufactured and used so as to comprising no cells and comprising decellularized extracellular matrix and alginate.

Specifically, bioink having a ratio of dECM and alginate obtained in Example 4 (mixed concentration ratio 1:1) was manufactured. Alginate 260 ul at a concentration of 2% by weight and dECM 200 ul at a concentration of 2.6% by weight, and cell culture medium 60 ul were prepared and were injected to a syringe with a 27 G nozzle. The pneumatic pressure was provided equally for 0.032, and spheroids were manufactured while changing the pneumatic pressure size of 5 kPa, 10 kPa to 100 kPa in 10 kPa increments. One hundred spheroids were manufactured in each experiment and the average shape was examined. After adding the mixed solution of 2 ml each of the calcium chloride solution (0.1 M) and Pluronic F-127 solution (20% by weight) to a 6-well plate, 100 per one well were manufactured while varying the pressure, and all experiment groups were manufactured only in 2 batches in total.

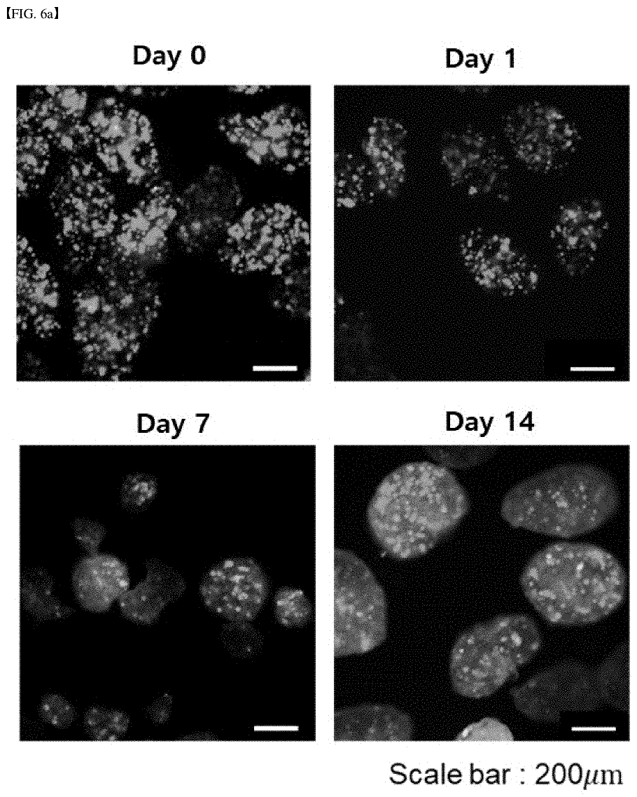

When applying the pneumatic pressure of 5 kPa, the pressure was weak and therefore the tail from the nozzle was formed and thus the spheroid shape was not homogeneous, and when applying the pneumatic pressure of 100 kPa, the spheroid had a too big size. Accordingly, when applying the pneumatic pressure of 30 kPa to 40 kPa, homogeneous spheroids having a size of 300 to 350 μm could be produced in quantity. FIG. 5 is a drawing which shows spheroids prepared by performing printing under various pneumatic pressure conditions according to one example of the present invention.

### Example 6

### Cell Viability Evaluation

The cell viability of cell spheroids manufactured in Example 1 was measured. Specifically, it was performed with 3 kinds of cells, that is, EPC only, CPC only, and mixed cells of mixing EPC and CPC at the same number ratio were used.

After preparing spheroids comprising each cell, a petri dish containing spheroids was transferred to a clean bench. To remove the mixed solution of calcium chloride (0.1 M) and Pluronic F-127 (20% by weight), the dish was cooled. When the viscosity of the mixed solution was lowered enough to flow, only the spheroids were filtered using a 100 um cell strainer. After two washes, the cell strainer comprising spheroids was placed on a 6-well plate. It was mixed to the cell culture medium so that the concentration of calcium chloride was 5 mM, and 5 ml each was put to each well so that the spheroids were sufficiently filled. Then, the cell culture medium was changed once a day. To see the viability, spheroids filtered with a sieve were added to a live/dead assay solution and were put in an incubator for 30 minutes, and then were observed with a fluorescent microscope.

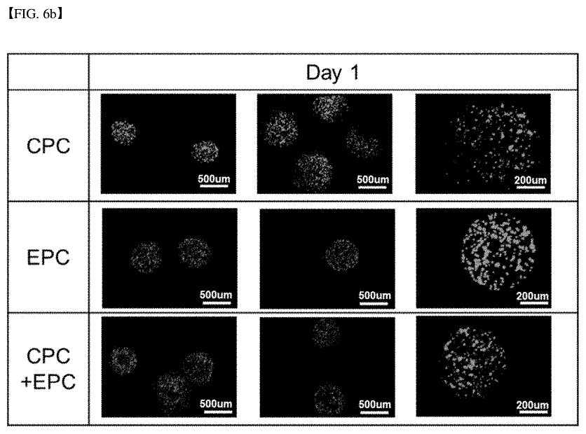

FIG. 6a is a photograph showing the viability of cells in the cell spheroid manufactured according to one example of the present invention, and FIG. 6b shows the viability at Day 1 as divided by cell. The viability at Day 1 and Day 7 showed that most of the cells were alive. Through this, it could be seen that the environment in the spheroid was made into a cell friendly niche environment.

1. A method of manufacturing a cell spheroid using a three-dimensional printing method, comprising
preparing a bioink comprising cells, decellularized extracellular matrix and alginate; and
manufacturing a cell spheroid by printing the bioink using the three-dimensional printing in a micro-extrusion manner.

2. The method of manufacturing according to claim 1, wherein manufacturing the spheroid comprises
inserting an nozzle to a mixed solution comprising a gelling agent of the alginate and hydrogel;
extruding the bioink by applying a pneumatic pressure; and
pulling out the injection nozzle from the mixed solution.

3. The method of manufacturing according to claim 2, further comprising applying high viscosity to the hydrogel by heating the mixed solution.

4. The method of manufacturing according to claim 2, wherein the mixed solution has enough high viscosity to form the cell spheroid by separating the extruded bioink from the injection nozzle with pulling out the injection nozzle.

5. The method of manufacturing according to claim 1, wherein the bioink is printed by being extruded at a pneumatic pressure of 30 kPa to 40 kPa.

6. The method of manufacturing according to claim 1, wherein the bioink comprises a decellularized extracellular matrix solution at a concentration of 0.5 to 5% by weight, and an alginate solution at a concentration of 0.5 to 5% by weight.

7. The method of manufacturing according to claim 1, wherein the alginate concentration of the bioink is equal to or more than the concentration of the decellularized extracellular matrix of the bioink.

8. The method of manufacturing according to claim 1, wherein the bioink comprises the decellularized extracellular matrix and the alginate at a concentration ratio of more than 1:0.5 to 1:5.

9. The method of manufacturing according to claim 1, wherein the bioink comprises the decellularized extracellular matrix solution and the alginate solution in a volume ratio of 1:0.33 to 1:3.

10. The method of manufacturing according to claim 1, wherein the cell spheroid has a diameter of 300 to 500 μm.

11. The method of manufacturing according to claim 1, wherein the bioink is extruded at a pneumatic-pressure time of 0.01 to 0.1 second using a 27 G nozzle, to form the spheroid having a diameter of 300 to 500 μm.

12. The method of manufacturing according to claim 1, wherein two or more of the cell spheroids are formed continuously as the bioink is sprayed with an injection nozzle and printed continuously, and located in circumstances of two or more concentric circles.

13. The method of manufacturing according to claim 1, wherein the manufacturing a cell spheroid by printing the bioink is performed by printing a cell spheroid with extruding the bioink by an injection nozzle moving along the spiral track.

14. The method of manufacturing according to claim 13, wherein the manufacturing a cell spheroid by printing the bioink comprises
determining information of a radius of a semicircle having the longest radius among the spiral tracks of the injection nozzle, the number of spheroids located in the circumference of the semicircle having the longest radius among the spiral tracks, a radius of a semicircle having the shortest radius among the spiral tracks, and an interval between circumferences of the spiral tracks;
determining the location at which the bioink is extruded by the injection nozzle using the determined information; and
manufacturing a plurality of cell spheroids continuously by printing the bioink on the determined locations.

15. The method of manufacturing according to claim 13, wherein the injection nozzle manufactures 100 or more of cell spheroids continuously.

16. The method of manufacturing according to claim 1, wherein the cell is at least one selected from the group consisting of cancer cell, stem cell, precursor cell, osteoblast, myoblast, tenocyte, neuroblast, fibroblast, glioblast, germ cell, hepatocyte, renal cell, Sertoli cell, chondrocyte, epithelial cell, cardiovascular cell, keratinocyte, smooth muscle cell, cardiocyte, cardiomyocyte, glial cell, endothelial cell, hormone-secreting cell, immunocyte, islet cell, pancreatic islet cell, neuron, thymocyte, adipocyte, alveolar cell, dental pulp cell, chondrocyte, oocyte and intestinal cell.

17. The method of manufacturing according to claim 16, wherein the stem cell is at least one selected from the group consisting of mesenchymal stem cell, induced pluripotent stem cell, induced pluripotent stem cell-based insulin producing cell, induced pluripotent stem cell-based cardiomyocyte, and induced pluripotent stem cell-based endothelial cell.

18. The method of manufacturing according to claim 1, wherein the cell spheroid is a drug testing model.

19. A composition for manufacturing a cell spheroid using a three-dimensional printing method, comprising cells, decellularized extracellular matrix and alginate as a gelated polymer.

20. The composition according to claim 19, wherein the concentration ratio of the decellularized extracellular matrix and the alginate is more than 1:0.5 or more to 1:5.
